(19) United States
(12) Patent Application Publication
(10) Pub. No.: US 2014/0303067 A1
(43) Pub. Date: Oct. 9, 2014

(54) TREATMENT OF EOSINOPHILIC ESOPHAGITIS

(71) Applicants: Johan Garssen, Nieuwegein (NL); Christophe Dupont, Clamart (FR); Catharina Theresia Knipping, Wezep (NL)

(72) Inventors: Johan Garssen, Nieuwegein (NL); Christophe Dupont, Clamart (FR); Catharina Theresia Knipping, Wezep (NL)

(73) Assignee: N.V. NUTRICIA

(21) Appl. No.: 14/349,651

(22) PCT Filed: Oct. 6, 2011
(86) PCT No.: PCT/NL2011/050677

§ 371 Date: Jun. 9, 2014

(51) Int. Cl. A61K 31/702; A61K 31/198; A61K 31/733; A61K 38/16

(52) U.S. Cl. CPC A61K 31/702; A61K 38/16; A61K 31/198; A61K 31/733
USPC 514/1.1; 514/23; 514/54

(57) ABSTRACT

The present invention provides a method for the treatment of eosinophilicesophagitis comprising administering non-digestible oligosaccharides.

### FIELD OF THE INVENTION

The present invention relates to a method for the treatment and/or prevention of eosinophilic esophagitis

### BACKGROUND OF THE INVENTION

Eosinophilic esophagitis (EoE) is characterized by an eosinophilic infiltration of the esophageal wall. EoE is recognized in children and adults and shows a predominance in males. Present treatments include dietary restrictions and corticosteroids.

WO 2008/015374 discloses a composition based on free amino acids without intact protein for treating amongst others EoE.

Neocate® Junior with Prebiotics is a commercially available nutritionally complete, amino acid-based medical food for children for the dietary management of allergy to food proteins and food-allergy-associated conditions including EoE. Neocate® Junior with Prebiotics contains prebiotic fibers inulin and short chain fructooligosaccharides to help promote digestive health especially helpful for children with GI-related malabsorptive conditions.

It has been suggested that immunoglobulin free light chains (Ig-fLC) might play a role in the development of mast cell mediated hypersensitivity-like responses, Kraneveld et al., Proc Natl Acad Sci USA 2005; 102:1578-83; Groot Kormelink et al., Clin Exp Allergy 2009; 39:33-42.

Concentrations of Ig-fLCs are low at birth and from the first week of life rapidly increase until low normal levels are attained by one year of age. From one year of age, a gradual increase in the concentration continues to the age of approximately 20 years. The concentrations Ig-fLCs are steady in the age group of 5-9 years, Solling, Scand J Clin Lab Invest 1977; 37:21-5.

### SUMMARY OF THE INVENTION

From a study on serum markers it was surprisingly found that in a subgroup of children diagnosed with eosinophilic esophagitis (EoE), the serum level of immunoglobulin free light chains (Ig-fLC) was elevated. Moreover, it was surprisingly found that serum Ig-fLC clearly was increased in females compared to males.

The identification of this new biomarker led to the notion that a therapy aimed at reducing immunoglobulin free light chain concentrations in plasma would be beneficial for this particular patient group of children suffering from EoE.

Recently it has been shown that non-digestible oligosaccharides reduce Ig-fLC plasma concentrations in infants at risk for allergies, Schouten et al. Pediatric Allergy and Immunology, 2011; 22:537-542.

Thus in view of the object of providing further therapies for individuals suffering from EoE, the present inventors found that administering non-digestible oligosaccharides meet this object. The present invention particularly aims at treatment and/or prevention or dietary management of individuals suffering from eosinophilic esophagitis by administering non-digestible oligosaccharides or administering a composition comprising non-digestible oligosaccharides to said individuals, particularly to those with increased serum Ig-fLC concentration and in particular to females in the age between 0 and 12 with increased serum Ig-fLC concentration.

### DETAILED DESCRIPTION OF THE INVENTION

The present invention provides a method for the treatment or dietary management of a human subject suffering from eosinophilic esophagitis (EoE) or a human subject at risk for eosinophilic esophagitis (EoE), comprising administering a composition comprising non-digestible oligosaccharides to the human subject wherein the human subject has an increased serum immunoglobulin free light chains (Ig-fLC) concentration.

The invention also concerns the use of a composition comprising non-digestible oligosaccharides for the manufacture of a composition for human subjects suffering from eosinophilic esophagitis and having an increased serum Ig-fLC concentration.

The invention can also be worded as a composition comprising non-digestible oligosaccharides for use in human subjects suffering from eosinophilic esophagitis and having an increased serum Ig-fLC concentration.

In other words the invention concerns the use of a composition comprising non-digestible oligosaccharides for the manufacture of a composition for the treatment and/or prevention or dietary management of eosinophilic esophagitis (EoE) in a human subject with an increased serum Ig-fLC concentration.

In other words the invention concerns a composition comprising non-digestible oligosaccharides for use in the treatment and/or prevention or dietary management of EoE in a human subject with an increased serum Ig-fLC concentration.

In one aspect, the present invention provides a method for the treatment or dietary management of a human subject suffering from eosinophilic esophagitis (EoE) or a human subject at risk for eosinophilic esophagitis (EoE), comprising administering a composition comprising non-digestible oligosaccharides and peptides to said human subject.

In other words the invention concerns the use of a composition comprising non-digestible oligosaccharides and peptides for the manufacture of a composition for the treatment and/or prevention or dietary management of eosinophilic esophagitis (EoE) in a human subject.

In other words the invention provides a composition comprising non-digestible oligosaccharides and peptides for use in the treatment and/or prevention or dietary management of EoE.

In the context of this invention the word ‘peptides’ refers to protein in the form of two or more linked amino acids. Thus ‘peptide’ includes partially hydrolyzed protein and intact protein. This definition is introduced here to distinguish from those instances in the present description where protein can refer exclusively to free amino acids, such as in the energy provided by the protein component and the source of protein.

In one aspect, the present invention concerns a method for the treatment or dietary management of a human subject suffering from eosinophilic esophagitis (EoE) or a human subject at risk for eosinophilic esophagitis (EoE), comprising administering non-digestible oligosaccharides to said human subject.

The invention also concerns the use of non-digestible oligosaccharides for the manufacture of a composition for the treatment and/or prevention or dietary management of eosinophilic esophagitis (EoE) in a human subject.

The invention can also be worded as non-digestible oligosaccharides for use in the treatment and/or prevention or dietary management of eosinophilic esophagitis (EoE) in a human subject.

As in general males are at higher risk for EoE, in one embodiment according to the present invention, the human subject is a male. According to the present invention, preferably the human subject has an increased serum Ig-fLC concentration.

As it was found that there is a predominance of females under the human subjects with EoE that have increased serum Ig-fLC, in a preferred embodiment according to the present invention, the human subject is a female, preferably a female with increased serum Ig-fLC.

The reduction of Ig-fLC plasma concentrations can best be achieved by providing a mixture of at least two non-digestible neutral oligosaccharides differing in structure and/or degree of polymerization (DP). As a mixture of non-digestible neutral oligosaccharides differing in structure and/or DP, galacto-oligosaccharides, and/or fructo-oligosaccharides are particularly suitable.

Thus in one aspect the present invention concerns a method for the treatment or dietary management of a human subject suffering from eosinophilic esophagitis (EoE) or a human subject at risk for eosinophilic esophagitis (EoE), comprising administering a combination of galacto-oligosaccharides and fructo-oligosaccharides to the human subject. Preferably the human subject has an increased serum Ig-fLC.

In other words the invention concerns the use of a combination of galacto-oligosaccharides and fructo-oligosaccharides for the manufacture of a composition for the treatment and/or prevention or dietary management of eosinophilic esophagitis (EoE) in a human subject.

The invention can also be worded as a combination of galacto-oligosaccharides and fructo-oligosaccharides for use in the treatment and/or prevention or dietary management of eosinophilic esophagitis (EoE) in a human subject.

In a further aspect the present invention can be suitably brought to practice by incorporation of the present active ingredients in a nutritional composition. Such composition can be administered to the human subject without posing a heavy burden on the human subject suffering from EoE or at risk of EoE.

The present invention preferably relates to the administration to humans having an age between 0 and 12 years, preferably having an age between 0 and 10 years, more preferably having an age between 0 and 8 years, more preferably having an age between 0 and 6 years, more preferably having an age between 0 and 3 years. At low age the impact of the method or use according to the invention is considered to be the highest.

Serum Immunoglobulin Free Light Chains (Ig-fLC)

In the context of this invention “serum immunoglobulin free light chains (Ig-fLC)” refers to the concentration of Ig-fLC in blood plasma of a human subject. The concentration of serum Ig-fLC can be determined by ELISA as specified in the examples. Preferably kappa Ig-fLC and lambda Ig-fLC are separately determined and in the context of the present invention, Ig-fLC is said to be increased if either one or both of kappa Ig-fLC and lambda Ig-fLC is/are increased.

Subjects with an increased concentration serum Ig-fLC are preferably those wherein the concentration serum kappa Ig-fLC is above 125% of the average concentration serum kappa Ig-fLC for the specific age in years, preferably above 150%; and/or wherein the lambda Ig-fLC is above 125% of the average concentration serum lambda Ig-fLC for the specific age (in years), preferably above 150%. Preferably the concentration serum lambda Ig-fLC and/or the concentration kappa Ig-fLC is above 21.8 μg/ml. A concentration of kappa Ig-fLC is preferably considered increased if this is above 21.8 μg/ml as determined by ELISA as specified in the examples and also a concentration of lambda Ig-fLC is considered increased if this is above 21.8 μg/ml as determined by ELISA as specified in the examples.

Non-Digestible Oligosaccharides

The term “non-digestible oligosaccharides” as used in the present invention refers to carbohydrates which are not digested in the intestine by the action of acids or digestive enzymes present in the human upper digestive tract (small intestine and stomach) but which are preferably fermented by the human intestinal microbiota. For example, sucrose, lactose, maltose and maltodextrins are considered digestible. The term “oligosaccharide” as used in the present invention refers to carbohydrates with a degree of polymerization (DP) of 2 to 250, preferably a DP 2 to 100, more preferably 2 to 60, even more preferably 2 to 10. If the oligosaccharide with a DP of 2 to 100 is included in the present preparation, this includes compositions which contain oligosaccharides with a DP between 2 and 5, a DP between 50 and 70 and a DP of 7 to 60.

Preferably the non-digestible oligosaccharides are soluble. The term “soluble” as used herein, when having reference to an oligosaccharide, means that the oligosaccharide is soluble according to the method described by L. Prosky et al., J. Assoc. Off. Anal. Chem. 71, 1017-1023 (1988).

Different non-digestible carbohydrates in the present invention relates to non-digestible carbohydrates differing in monosaccharide unit composition, or differing in degree of polymerization (DP) or both. Two non-digestible carbohydrates differ in monosaccharide composition when there is at least 30 mol % difference, more preferably at least 50 mol % difference in monosaccharide composition based on total mol monosaccharide units.

For instance galacto-oligosaccharides with an average composition of Glu-Gal3 and fructo-oligosaccharides with an average composition of Glu-Fru3 differ for 75 mol %. Two non-digestible carbohydrates differ in DP if the average DP of the two carbohydrates differs more than 5 monosaccharide units, preferably more than 10 units, even more preferably more than 15 units. For example hydrolysed inulin with an average DP of 4 and long chain inulin with an average DP of 25 have a difference in DP of 21 units.

For further improvement, the present non-digestible oligosaccharides preferably have a relatively high content of short chain oligosaccharides. Hence, preferably at least 10 wt. % of the non-digestible oligosaccharides has a DP of 2 to 5 (i.e. 2, 3, 4, and/or 5) and at least 5 wt. % has a DP of 10 to 60. Preferably at least 50 wt. %, more preferably at least 75 wt. % of the non-digestible oligosaccharides has a DP of 2 to 9 (i.e. 2, 3, 4, 5, 6, 7, 8, and/or 9).

Preferably the non-digestible oligosaccharides comprise galacto-oligosaccharides. The galacto-oligosaccharides are preferably selected from the group consisting of beta-galacto-oligosaccharides. In a particularly preferred embodiment the present preparation comprises beta-galacto-oligosaccharides. Beta-galacto-oligosaccharides as used in the present invention refers to oligosaccharides composed of over 50%, preferably over 65% galactose units based on monomeric subunits, with a degree of polymerization (DP) of 2 to 20, in which at least 50%, more preferably at least 75%, even more preferably at least 90%, of the galactose units are linked together via a beta-glycosidic linkage, preferably a beta-1,4 glycosidic linkage. The average DP is preferably in the range of 3 to 6. A glucose unit may be present at the reducing end of the chain of galactose units. Beta-galacto-oligosaccharides are sometimes also referred to as transgalacto-oligosaccharides (TOS). A suitable source of beta-galacto-oligosaccharides is Vivinal®GOS (commercially available from Borculo Domo Ingredients, Zwolle, Netherlands). Other suitable sources are Oligomate (Yakult), Cupoligo, (Nissin) and Bi2muno (Classado).

Preferably the non-digestible oligosaccharides comprise fructo-oligosaccharides. Fructo-oligosaccharides as used in the present invention refers to oligosaccharides composed of over 50%, preferably over 65% fructose units based on monomeric subunits, in which preferably at least 50%, more preferably at least 75%, even more preferably at least 90%, of the fructose units are linked together via a beta-glycosidic linkage, preferably a beta-2,1 glycosidic linkage. A glucose unit may be present at the reducing end of the chain of fructose units. Preferably the fructo-oligosaccharide has a DP or average DP in the range of 2 to 250, more preferably 2 to 100, even more preferably 10 to 60. Fructo-oligosaccaride comprises levan, hydrolysed levan, inulin, hydrolysed inulin, and synthesised fructo-oligosaccharides. Preferably the non-digestible oligosaccharides comprise short chain fructo-oligosaccharides with an average degree of polymerization (DP) of 3 to 10, more preferably hydrolysed inulin or synthetic fructo-oligosaccharide. Fructo-oligosaccharide suitable for use according to the present invention is also readily commercially available, e.g. RaftilineHP (Orafti). Preferably the non-digestible oligosaccharides comprise long chain fructo-oligosaccharides with an average DP between 10 and 60. Preferably the non-digestible oligosaccharides comprise both short chain and long chain fructo-oligosaccharides. Hence, in one embodiment, the non-digestible oligosaccharides comprise short-chain fructo-oligosaccharides with an average degree of polymerization (DP) of 3 to 10 and long chain fructo-oligosaccharides with an average DP between 10 and 60. Preferably the weight ratio short chain fructo-oligosaccharides:long chain fructo-oligosaccharides is in the range of 1:1 to 20:1, preferably in the range of 2:1 to 15:1, preferably in the range of 4:1 to 12:1, preferably in the range of 5:1 to 10:1, more preferably about 9:1.

More preferably the non-digestible oligosaccharides comprise a combination of galacto-oligosaccharides and fructo-oligosaccharides. More preferably the non-digestible oligosaccharides comprise a combination of galacto-oligosaccharides with an average DP in the range of 2-10, preferably in the range of 2-7 and fructo-oligosaccharides. More preferably the non-digestible oligosaccharides comprise a combination of galacto-oligosaccharides and fructo-oligosaccharides with an average DP in the range of 10-100, preferably in the range of 10-60, more preferably in the range of 20-60. More preferably the non-digestible oligosaccharides comprise a combination of galacto-oligosaccharides with an average DP in the range of 2-10, preferably in the range of 2-7 and fructo-oligosaccharides with an average DP in the range of 10-100, preferably in the range of 10-60, more preferably in the range of 20-60.

Preferably the weight ratio galacto-oligosaccharides : fructo-oligosaccharides is in the range of 1:1 to 20:1, preferably in the range of 2:1 to 15:1, preferably in the range of 4:1 to 12:1, preferably in the range of 5:1 to 10:1, more preferably about 9:1.

The present method preferably comprises the administration of a serving comprising between 0.05 and 25 grams non-digestible oligosaccharide, preferably between 0.1 and 5 grams. The present method preferably comprises the administration of a serving comprising between 0.05 and 25 grams galacto-oligosaccharides, preferably between 0.1 and 5 gram galacto-oligosaccharides.

Formulae

The present composition is preferably enterally administered, more preferably orally.

The present composition is preferably a nutritional formula, preferably a formula for children, preferably infants. In the context of this invention, children are defined as having an age of 0 to 14 years. Children with an age of 0 to 3 years can also be referred to as infants. The present composition can be advantageously applied as a complete nutrition for children. The present composition preferably comprises a lipid component, protein component and carbohydrate component and is preferably administered in liquid form. The present composition can also be in the form of dry food (e.g. powders) which is accompanied with instructions as to admix said dry food with a suitable liquid (e.g. water).

According to the present invention, preferably the composition comprises a lipid component that provides 5 to 50% of the total calories, a protein component that provides 5 to 50% of the total calories, and a digestible carbohydrate component that provides 15 to 90% of the total calories. Preferably, the lipid component provides 35 to 50% of the total calories, the protein component provides 7.5 to 12.5% of the total calories, and the carbohydrate component provides 40 to 55% of the total calories. For calculation of the % of total calories, the total of energy provided by the protein component, digestible carbohydrate component and lipid component needs to be taken into account. For calculation of the % of total calories for the protein component, the total of energy provided by the proteins, any form of protein, including peptides and amino acids, needs to be taken into account.

The present composition preferably comprises at least one lipid selected from the group consisting of animal lipid (excluding human lipids) and vegetable lipids. Preferably the present composition comprises a combination of vegetable lipids and at least one oil selected from the group consisting of fish oil, animal oil, algae oil, fungal oil, and bacterial oil. The present composition comprising non-digestible oligosaccharides excludes human milk.

In one embodiment according to the invention, the composition further comprises peptide, e.g. protein in the form of two or more linked amino acids. In one embodiment according to the invention, the composition further comprises intact protein. In one embodiment the protein in the form of two or more linked amino acids or the intact protein is selected from the group consisting of non-human animal proteins (preferably milk proteins) and vegetable proteins (preferably soy protein and/or rice protein). For example, the present composition preferably contains casein, whey protein and/or vegetable protein or peptide.

Human subjects suffering from EoE or who are at risk of EoE benefit from nutrition that is at low risk of eliciting an allergic response. Therefore in one embodiment according to the invention, the composition further comprises partially hydrolysed protein. Thus in one embodiment according to the invention the composition preferably contains hydrolysed casein and/or hydrolysed whey protein.

More advantageously, in one embodiment according to the invention, the composition does not comprise intact protein and also does not comprise partially hydrolysed protein. Preferably the composition comprises free amino acid as the sole source of protein.

According to the invention, the digestible carbohydrates are preferably selected from the group consisting of sucrose, lactose, glucose, fructose, corn syrup solids, starch and maltodextrins, more preferably lactose. For sensitive subjects preferably all ingredients causing allergic reaction or potential discomfort are removed. Hence, in one embodiment, preferably the present composition does not contain lactose.

### EXAMPLES

### Example 1

Method

The study was carried out in a population of children undergoing a clinical and biological evaluation for EoE. The study population (28 children) was composed of 21 boys (age 6.7 years±4.2 SD) and 7 girls (age 8.2 years±5.6 SD) suffering from EoE demonstrated by esophageal biopsy showing a number of eosinophils>30/high power field (HPF).

Serum Immunoglobulin Free Light Chain (Ig-fLC)

Total kappa and lambda Ig-fLC serum concentrations were determined using an ELISA adapted from Abe et al. Clin Exp Immunol 1998; 111:457-62. Reference levels of Ig-fLC were obtained in a cohort of 250 children (data not shown) with different allergic manifestations. Ig-fLC concentrations above average +SD (for both kappa and lambda Ig-fLC 21.8 μg/ml) were considered elevated.

Results

The results are provided in Table 1.

Serum Immunoglobulin Free Light Chain (Ig-fLC)

Based on the reference values provided in the method section, in the total population elevated levels were found for both kappa and lambda Ig-fLC in 6/28 (21.4%) of the children.

These results were split according to gender distribution. Total levels of both kappa and lambda Ig-fLC were significantly higher in females than males (kappa Ig-fLC 14.3 (male) versus 21.7 (female) μg/ml, p=0.04 ; lambda Ig-fLC 13.9 (male) versus 22.0 (female) μg/ml, p=0.001).

Elevated kappa Ig-fLC were seen in 2/21 (9.5%) males versus 4/7 (57.1%) females (p=0.008) and elevated lambda Ig-fLC in 1/21 (4.8%) males versus 5/7 (71.4%) females, (p=<0.001).

### CONCLUSION

Blood Ig-fLC appeared clearly increased in females as compared to males, thereby adding another gender difference in the biology of this disease. Considering the sex differences observed in EoE, these results suggest that Ig-fLC might act as an agonist allowing the disease to occur in females, who are obviously less prone to develop the disease.

TABLE 1
Clinical symptoms of the study population and
biological parameters.

| Date of birth | M/F | kappa Ig-fLC (μg/ml) | lambda Ig-fLC (μg/ml) |
|---|---|---|---|
| 24-2-1996 | M | 25.0 | 15.9 |
| 4-12-2000 | M | 14.9 | 19.2 |
| 28-6-2002 | M | 14.6 | 11.0 |
| 16-11-2001 | M | 19.3 | 15.0 |
| 16-12-1995 | M | 14.4 | 12.7 |
| 8-9-2006 | M | 11.0 | 11.6 |
| 11-8-1995 | M | 18.4 | 14.2 |
| 14-12-2005 | M | 7.6 | 10.3 |
| 13-3-2001 | M | 11.2 | 12.1 |
| 25-5-2008 | M | 5.6 | 9.6 |
| 8-5-2001 | M | 10.5 | 18.0 |
| 24-8-2006 | M | 3.1 | 6.3 |
| 23-9-1996 | M | 7.4 | 11.9 |
| 1-11-2006 | M | 12.3 | 19.6 |
| 28-9-2002 | M | 10.1 | 9.1 |
| 16-9-2002 | M | 14.4 | 12.4 |
| | M | 15.8 | 10.2 |
| 22-9-2004 | M | 16.4 | 11.6 |
| 9-12-2008 | M | 33.2 | 19.7 |
| 6-10-1996 | M | 19.4 | 18.7 |
| 3-11-2009 | M | 16.8 | 23.5 |
| Average | | 14.3 | 13.9 |
| % elevated | | 9.5 | 4.8 |
| 24-8-2005 | F | 9.7 | 12.4 |
| 25-8-1996 | F | 10.0 | 13.5 |
| 19-5-2001 | F | 21.9 | 24.2 |
| 29-1-2000 | F | 29.5 | 23.2 |
| 19-4-1993 | F | 36.7 | 24.4 |
| 4-5-2004 | F | 17.0 | 23.2 |
| 17-5-2009 | F | 27.0 | 33.3 |
| Average | | 21.7 | 22.0 |
| % elevated | | 57.1 | 71.4 |

M = male;
F = female;
Ig-fLC; immunoglobulin free light chain;
concentrations that are considerd increased are in bold type face

### Example 2

Nutrilon™ Hypo-Allergeen (10% of total calories protein, 47% of total calories lipid, carbohydrates 43% of total calories) with galactooligosaccharides and fructooligosaccharides which is indicated for the dietary management of EoE in female infants with the age between 0 and 3 years.

### Example 3

Neocate™ which contains per 100 gram powder: 13 g amino acids, 54 g digestible carbohydrates, 23 g lipid and fructooligosaccharides and inulin as non-digestible oligosaccharides, indicated for dietary management of infants suffering from EoE with the age between 1 and 3 years, wherein said infants have an increased serum concentration of immunoglobulin free light chains, particularly increased lambda Ig-fLC and/or kappa Ig-fLC.

1-15. (canceled)

16. A method for the treatment and/or prevention of eosinophilic esophagitis, comprising administering to a human subject having an increased serum immunoglobulin free light chains (Ig-fLC) concentration a composition comprising non-digestible oligosaccharides.

17. The method according to claim 16, wherein the human subject is a child having an age of 0 to 12 years.

18. The method according to claim 16, wherein the human subject is female.

19. The method according to claim 16, wherein the composition further comprises intact protein.

20. The method according to claim 16, wherein the composition further comprises partially hydrolysed protein.

21. The method according to claim 16, wherein the composition further comprises free amino acids without intact or partially hydrolysed protein.

22. The method according to claim 16, wherein the non-digestible oligosaccharides are selected from one or more from the group consisting of fructo-oligosaccharides and galacto-oligosaccharides.

23. The method according to claim 16, wherein the non-digestible oligosaccharides comprise short chain fructo-oligosaccharides and long chain fructo-oligosaccharides.

24. The method according to claim 16, wherein the non-digestible oligosaccharides comprise galacto-oligosaccharides and fructo-oligosaccharides.

25. The method according to claim 16 wherein the composition comprises:
(a) a lipid component, which provides 5 to 50% of the total calories,
(b) a protein component, which provides 5 to 50% of the total calories, and
(c) a carbohydrate component, which provides 15 to 90% of the total calories.

26. A method for the treatment and/or prevention of eosinophilic esophagitis, comprising administering to a human subject in need thereof a composition comprising non-digestible oligosaccharides and peptides.

27. The method according to claim 26, wherein the non-digestible oligosaccharides comprise fructooligosaccharides.
